(19) United States
(12) Patent Application Publication
(10) Pub. No.: US 2013/0079637 A1
(43) Pub. Date: Mar. 28, 2013

(54) ULTRASOUND IMAGING ACOUSTIC INFORMATION PRESENTATION

(71) Applicant: Peter Ulrik Kann, Farum (DK)

(72) Inventor: Peter Ulrik Kann, Farum (DK)

(73) Assignee: B-K MEDICAL APS, Herlev (DK)

(21) Appl. No.: 13/245,621

(22) Filed: Sep. 26, 2011

(51) Int. Cl. A61B 8/14

(52) U.S. Cl. 600/443

(57) ABSTRACT

An ultrasound imaging system includes receive circuitry configured to receive an ultrasound echo from structure in a medium being scanned with the ultrasound imaging system, an acoustic processor configured to process the ultrasound echo and generate an acoustic signal indicative of the ultrasound echo, an acoustic output device configured to acoustically present the acoustic signal, and an acoustic diffuser configured to receive and diffuse the presented acoustic signal.

### TECHNICAL FIELD

The following generally relates to ultrasound imaging and more particularly to an ultrasound imaging system configured to produce and output acoustic information such as Doppler information and/or other acoustic information and controllably diffuse the output acoustic information.

### BACKGROUND

Ultrasound imaging has provided useful information about the interior characteristics of an object or subject under examination.

An ultrasound imaging system has included a probe with a transducer array that is configured to transmit an ultrasound signal into the object or subject under examination. As the signal traverses the object or subject, portions of the signal are attenuated, scattered, and/or reflected off structure in the object or subject, with some of the reflections traversing back towards the transducer array. The later reflections are referred to as echoes. The transducer array is further configured to receive the echoes.

In B-mode imaging, the received echoes correspond to a two dimensional (2D) slice (e.g., an axial, sagittal, etc. slice) through the object or subject and are processed to generate scanlines, which are used to generate a scanplane, or two dimensional image of the slice or plane, which can be displayed via a monitor, display or the like. A three-dimensional (3D) image can be created from a series of adjacent two dimensional images. B-mode scanplanes have been combined with color flow, Doppler flow, and/or other information.

Doppler ultrasound can be used to acoustically image flow. Generally, Doppler ultrasound employs the Doppler Effect to determine the direction of flow of a flowing structure and/or a relative velocity of the flowing structure in a tubular entity. In medical imaging application, the structure has included blood cells where the tubular entity has included arteries, veins and the like. The Doppler information can be visualized in a graph of velocity as a function of time, visualized as a color overlay superimposed over an image, audibly presented in an acoustic signal and/or otherwise presented.

FIG. 1 shows an example of audibly presenting Doppler information as an acoustic signal. In FIG. 1, an ultrasound imaging system 100 includes a console 102 with a display region 104 and a first speaker 106 and a second speaker 108 in a stereo arrangement. That is, the first speaker 106 is located to a first side 110 of the display region 104 and the second speaker 108 is located on a second side 112 of the display region 104, with both speakers 106 and 108 facing out from a direction of a plane of the display region 104.

In the illustrated embodiment, the speakers 106 and 108 are located on a support 114 such as a desk, cart, etc., which is shared by the console 102. Alternatively, the speakers 106 and 108 can be affixed to the sides 110 and 112 of the console 102 and/or other support. In this arrangement, Doppler information is acoustically presented using the speaker 106 and 108. Unfortunately, the acoustic information perceived by an entity such as an operator of the system 100 depends on the entity's spatial position to the speakers 106 and 108.

By way of example, FIGS. 2 and 3 respectively show a top down view and a side view of FIG. 1 in which a first entity 202 is located relative to the speakers 106 and 108 so as to perceive a similar or same frequency response and sound pressure level from the two speakers 106 and 108 (e.g., a stereo acoustical image of interest), and a second entity 204 is located relative to the speakers 106 and 108 so as to perceive a different acoustical image from the two speakers 106 and 108, with the contribution from each speaker 106 and 108 also being different.

Unfortunately, in both FIGS. 2 and 3, the second entity 204 does not perceive the stereo acoustical image of interest received by the first entity 202. That is, the second entity 204 perceives different frequency responses from the speakers 106 and 108 and different sound pressure levels from the speakers 106 and 108. In addition, with the speaker arrangement of FIG. 1, in which the speakers 106 and 108 face forward, the speaker cover or grill is susceptible to debris and/or contamination, and can be difficult to clean. Furthermore, the cover or grill takes up space and/or can be difficult to integrate into the ultrasound console 100.

### SUMMARY

Aspects of the application address the above matters, and others.

In one aspect, an ultrasound imaging system includes receive circuitry configured to receive an ultrasound echo from structure in a medium being scanned with the ultrasound imaging system, an acoustic processor configured to process the ultrasound echo and generate an acoustic signal indicative of the ultrasound echo, an acoustic output device configured to acoustically present the acoustic signal, and an acoustic diffuser configured to receive and diffuse the presented acoustic signal.

In another aspect, a method includes processing, via an acoustic processor, an ultrasound echo and generating an acoustic signal indicative of the ultrasound echo, outputting the acoustic signal, and receiving and controllably diffusing, via an acoustic diffuser, the output acoustic signal.

In another aspect, a method includes diffusing ultrasound Doppler acoustic information output by an ultrasound imaging system such that at least one of a frequency response and a sound pressure level is the same or substantially the same throughout a predetermined audible information receiving zone.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically illustrates a prior art ultrasound imaging system which uses speakers to audibly present Doppler ultrasound information to an operator of the ultrasound imaging system;

FIG. 2 schematically illustrates a top down view of the ultrasound imaging system of FIG. 1, showing how the location of the operator with respect to the speakers impacts the acoustic information received by the operator;

FIG. 3 schematically illustrates a side view of the ultrasound imaging system of FIG. 1, showing how the location of the operator with respect to the speakers impacts the acoustic information received by the operator;

FIG. 4 schematically illustrates an ultrasound imaging system that includes an acoustic diffuser which diffuses acoustic information output by the ultrasound imaging system;

FIG. 5 illustrates an example of the ultrasound system of FIG. 4;

FIG. 6 schematically illustrates a side view of the example ultrasound system of FIG. 5, showing an example of the acoustic diffuser;

FIG. 7 schematically illustrates a top down view of the example ultrasound system of FIG. 5, showing an example of the acoustic diffuser;

FIG. 8 schematically illustrates a variation of the configuration of FIG. 5 in which the acoustic information is acoustically presented in a direction up from the console;

FIG. 9 schematically illustrates a variation of the configuration of FIG. 5 in which the acoustic information is acoustically presented in a direction up down from the console;

FIG. 10 schematically illustrates a variation of the configuration of FIG. 5 in which the acoustic information is acoustically presented to the sides of the console;

FIG. 11 schematically illustrates a variation of the configuration of FIG. 5 in which the acoustic information is acoustically presented to the diagonal from the console;

FIG. 12 schematically illustrates a variation of the configuration of FIG. 5 in which the acoustic information is acoustically presented in a direction up from the console and in a direction down from the console;

FIG. 13 schematically illustrates a variation of the configuration of FIG. 5 in which the acoustic information is acoustically conveyed from the top to the bottom of the console using sound pipes; and

FIG. 14 schematically illustrates a method.

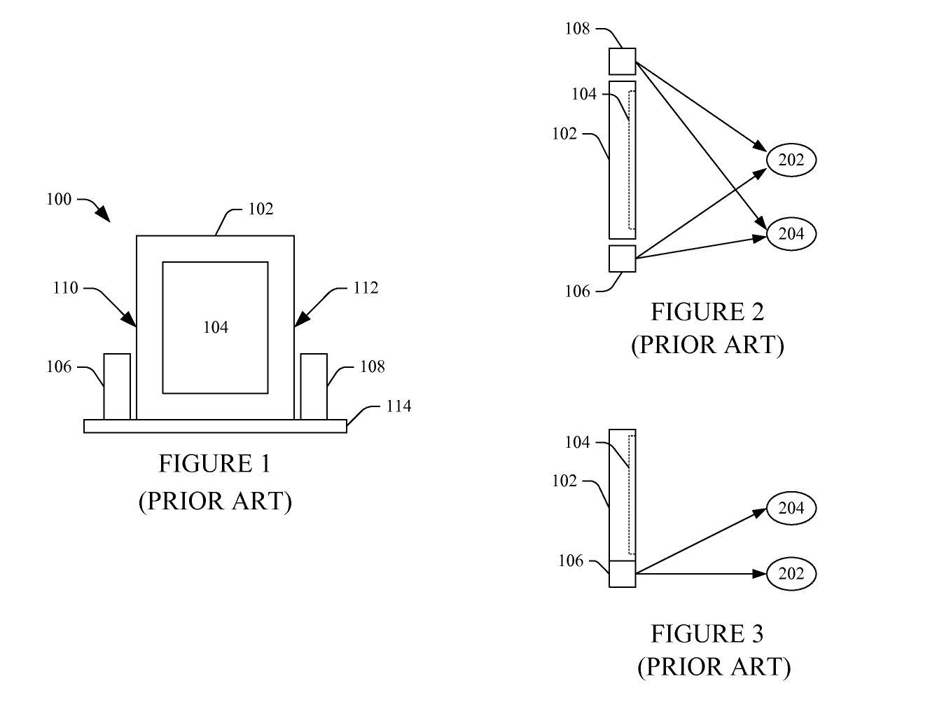

### DETAILED DESCRIPTION

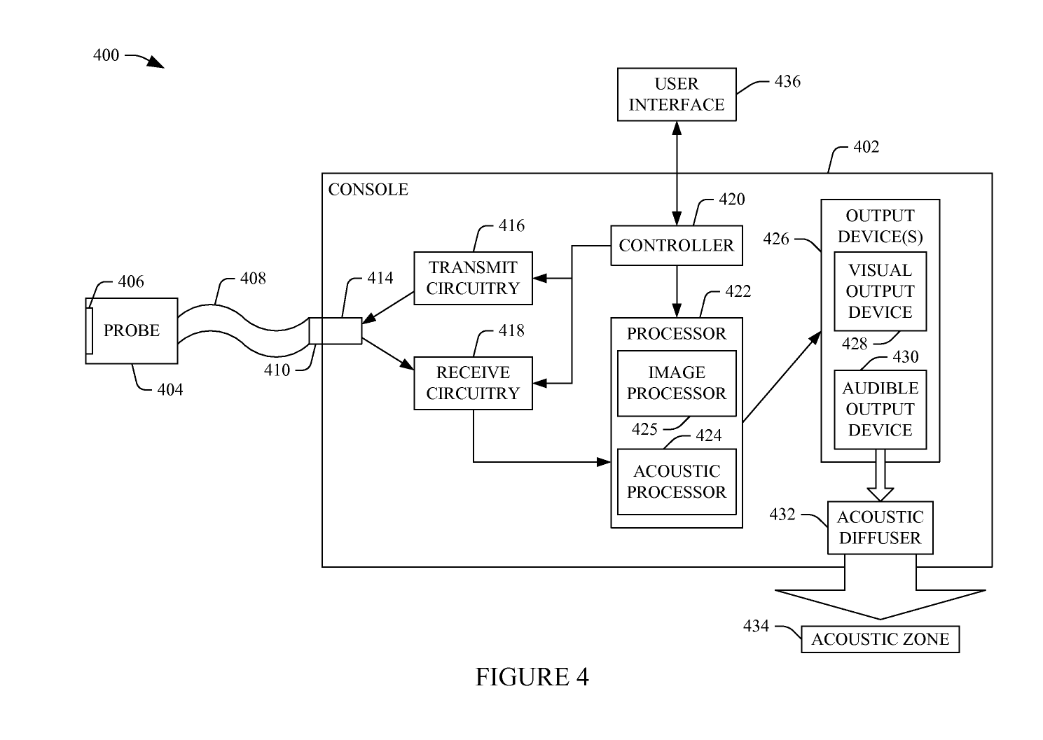

FIG. 4 schematically illustrates an imaging system 400, such as an ultrasound imaging system or scanner, which includes a console 402 and a probe 404 in communication therewith.

The probe 404 includes one or more transducer arrays 406, each having one or more transducer elements such as 16, 64, 128, 196, 256, etc. elements. The individual arrays may include linear, curved, and/or rotary transducer arrays, and the different arrays can be employed individually, simultaneously or in an interleaved manner to acquire data. The elements are configured to be driven in a frequency range of 1 to 18 MHz and/or other frequency range. The probe 404 also includes a cable 408 and a connector 410.

The console 402 includes a connector 414, which is configured to electrically and mechanically interface with the connector 410 of the probe 404 and/or a complementary connector of another probe. In the illustrated embodiment, the connector 414 is shown electrically and mechanically interfaced with the connector 410 of the probe 404. The console 402 and probe 404 communicate with each other via the connection between the connectors 410 and 414.

The console 402 further includes transmit circuitry 416 and receive circuitry 418. The transmit circuitry 416 generates electrical signals which control transducer element phasing and/or time of actuation, which allows for steering and/or focusing an ultrasound beam from predetermined origins and/or at predetermined angles, pulsing the signals, etc. The receive circuitry 418 receive ultrasound echoes. A controller 420 controls the transmit circuitry 416 and the receive circuitry 418.

A processer 422 processes the echoes received by the receive circuitry 418. The illustrated processor 422 at least includes an acoustic processor 424 such as a Doppler and/or other processor, which can process echoes at least based on the Doppler Effect to determine flow information (e.g., existence, direction, change in direction, etc.) about moving structure and/or a relative velocity of the moving structure. By way of example, the acoustic processor 424 can calculate the frequency shift of sample volume (e.g., blood) flow in a tubular structure (e.g., an artery, a vein, a chamber of the heart, etc.) and, from this, determine the speed and direction of the sample volume.

Optionally, the processer 422 can include one or more other processors such as an image processor 425, which is configured for other processing such as A-mode processing in which a single element scans a line through the body with the echoes plotted on screen as a function of depth, B-mode processing in which multiple element scan a plane that can be viewed as a two-dimensional (2D) image, C-mode processing in which an image is formed in a plane normal to a B-mode image, and/or other known processing and/or other processing and/or combinations of processing.

One or more output devices 426 can be employed to output the processed data. By way of non-limiting example, a visual output device 428 can be used to visually present Doppler data. For instance, the Doppler and/or other data can be visualized in a graph of velocity as a function of time, as a color overlay superimposed over an image via a visual output device 428 such as a computer display or monitor, and/or otherwise. By way of another example, an audible output device 430 can be used to audibly present the acoustic (e.g., Doppler data) and/or other acoustic data. For instance, the acoustic data can be audibly presented via audible output device 430 such as one or more acoustic speakers.

An acoustic diffuser 432 is configured to diffuse acoustic information such as Doppler data presented by the audible output device 430. As described in greater detail below, in one instance, the acoustic diffuser 432 diffuses acoustic information such that the acoustic information has a substantially similar or same acoustic property (e.g., frequency response (e.g., +10 kHz) and/or sound pressure level) throughout a predetermined audible information receiving zone (acoustic zone) 434 of the ultrasound system 400, which, generally, represents a physical area in the examination room where a clinician or other entity might be, in relation to the ultrasound system 400, to perform and/or receive diagnostic information acoustically presented by the ultrasound system 400.

A user interface 436 includes various input and/or output components, which allow a user to interact with the ultrasound system 400. Examples of such interaction include, but are not limited to, allowing a user to select an imaging protocol (e.g., Doppler ultrasound), select a presentation mode (e.g., visual and/or audible), initiate scanning, visually observe graphically presented information, audibly observe audibly presented information. The user interface 436 may include various buttons, knobs, keys, switches, touch sensitive areas, etc. for such interaction.

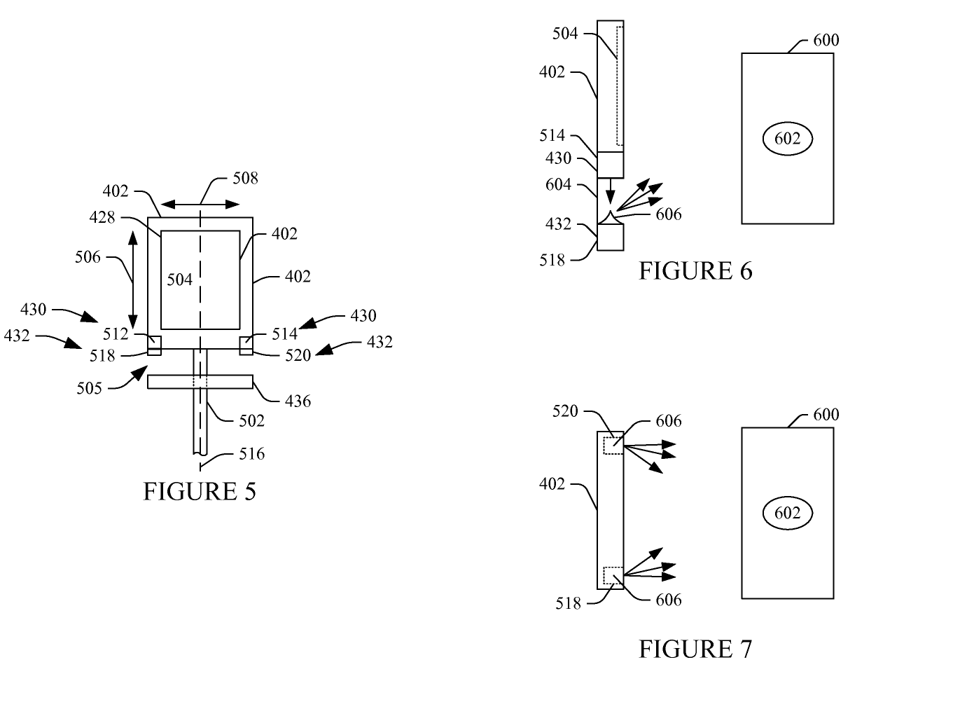

FIG. 5 illustrates a non-limiting example of the console 402 and the user interface 436 in connection with a support 502, and FIGS. 6 and 7 schematically illustrate the console 402 in connection with a predetermined audible information receiving zone 600.

Initially referring to FIG. 5, the visual output device 428 includes a two dimensional (2D) display 504 having a height 506 and a width 508. In this example, the console 402 is affixed to the support 502 such that the display 504 is arranged in a portrait orientation (i.e., the height 506 is greater than width 508), with the height 506 extending in a generally vertical direction and the width 508 extending in a generally horizontal direction, with respect to the examination room floor.

In another embodiment, the console 402 is affixed to the support 502 such that the display 504 is arranged in a landscape orientation (i.e., the height 506 is less than width 508), with the height 506 extending in a generally horizontal direction with respect to the examination room floor and the width 508 extending in a generally vertical direction with respect to the examination room floor. In yet another embodiment, the height 506 and the width 508 are equal in size.

The user interface 436 is affixed to the support 502 such that it is located below a bottom 505 of the console 402, between the console 402 and the examination room floor, and extends or protrudes in a generally horizontal direction away from a visual presentation region of the display 504.

The audible output device 430 includes acoustic speakers 512 and 514, which are arranged in a stereo configuration in which the acoustic speakers 512 and 514 are located on opposite sides of an imaginary vertical line 516, bisecting the width 508. Furthermore, the illustrated speakers 512 and 514 are spatially oriented such that they present audible information in a direction towards the examination room floor. With the speakers 512 and 514 facing down, a speaker grill, which can be difficult to clean, takes up space, and/or can be difficult to integrate into the design, can be mitigated. The speakers 512 and 514 can be located in the console 402 (as shown) or in connection with the outside of the console 402.

The acoustic diffuser 432 includes acoustic diffusers 518 and 520, with, in the illustrated embodiment, the acoustic diffuser 518 being located below the speaker 512, and the acoustic diffuser 520 being located below the speaker 514. In this example, the acoustic diffusers 518 and 520 are affixed to the console 402 and are configured to receive and diffuse the audible information from the speakers 512 and 514.

FIG. 6 schematically illustrates a side view of the example configuration of FIG. 5, in connection with the predetermined audible information receiving zone 600. In this embodiment, the acoustic diffuser 432 is affixed to the console via support structure 604. In another embodiment, the acoustic diffusers 432 are affixed to support structures that are separate from the imaging system 400.

The acoustic diffuser 432 includes a diffusion element 606 which diffuses the acoustic information output by the audible output device 430. It is to be appreciated that the geometry of the illustrated diffusion element 606 and/or the diffuser 432 are provided for explanatory purposes and are not limiting. As such, it is to be understood that diffusion element 606 and/or diffuser 432 with other geometry are also contemplated herein.

The predetermined audible information receiving zone 600 represents a physical area in the examination room where a clinician or other entity might be, in relation to the ultrasound system 400, to perform and/or receive diagnostic and/or other information of interest acoustically presented from the audible output device 430 in connection with an imaging procedure.

As discussed above, the clinician and/or other entity 602, in the predetermined audible information receiving zone 600, generally, perceives a similar or same acoustic characteristic such as a frequency response and/or sound pressure level from the audible output device 430 regardless of where the clinician and/or other entity 602 is in the zone 600. In the illustrated embodiment, the clinician and/or other entity 602 are located at a center region of the zone 600.

FIG. 7 schematically illustrates a top down view of the example configuration of FIG. 5, in connection with the predetermined audible information receiving zone 600.

With FIG. 7, like FIG. 6, the clinician and/or other entity 602, generally, perceives a similar or same acoustic characteristic such as a frequency response and/or sound pressure level from the audible output device 430 regardless of where the clinician and/or other entity 602 is in the zone 600.

Examples of suitable acoustic diffusers 432 include, but are not limited to, maximum length sequence diffusers, quadratic-residue diffusers, primitive root diffusers, optimized diffusers, two dimensional diffusers and/or other acoustic diffusers.

Variations are contemplated.

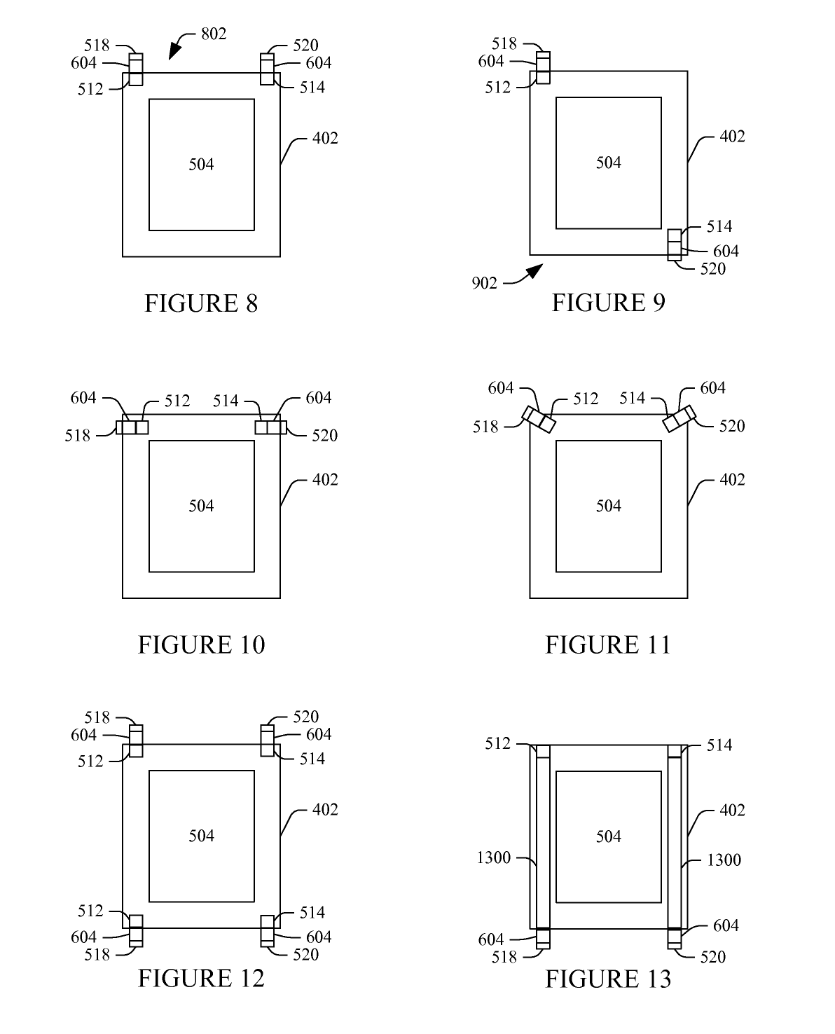

In FIG. 8, the acoustic speakers 512 and 514 are alternatively located at a top region 802 of the console 402 and oriented so that they present acoustic information in the opposite direction as that in FIG. 5, and the acoustic diffusers 518 and 520 are arranged above the speakers 512 and 514, likewise, to receive and diffuse the acoustic information to the zone 600.

In FIG. 9, the acoustic speaker 512 is located at the top region 802 of the console 402 and the acoustic speaker 514 is located at a bottom region 902 of the console 402. Again, the acoustic diffusers 518 and 520 are arranged above the speakers 512 and 514, likewise, to receive and diffuse the acoustic information to the zone 600.

In FIG. 10, the acoustic speakers 512 and 514 are located at the top region 802 of the console 402 and oriented so that they present acoustic information in the opposite direction as that in FIG. 5, and the acoustic diffusers 518 and 520 are arranged with respect to the speakers 512 and 514, likewise, to receive and diffuse the acoustic information to the zone 600.

Similar to FIGS. 5, 8, and 9, in FIG. 10 the acoustic speakers 512 and 514 can alternatively be located at the bottom region 902 of the console 402 to receive and diffuse the acoustic information to the zone 600 or two sets can be included, one at the top region 802 and one at the bottom region.

In FIG. 11, the acoustic speakers 512 and 514 are oriented so that they present acoustic information in a diagonal direction with respect to the display 504, and the acoustic diffusers 518 and 520 are arranged above the speakers 512 and 514, likewise, to receive and diffuse the acoustic information to the zone 600.

Similar to FIGS. 5, 8, and 9, in FIG. 11 the acoustic speakers 512 and 514 can alternatively be located at the bottom region 902 of the console 402 to receive and diffuse the acoustic information to the zone 600 or two sets can be included, one at the top region 802 and one at the bottom region.

FIG. 12 shows a variation which is the combination of FIGS. 5 and 8.

In FIG. 13, acoustic pipes 1300 are used to convey the acoustics information from the speakers 512 and 514 located at the top region 802 of the console 402 to the acoustic diffusers 518 and 520 which are located at the bottom region 902 of the console 402 and receive and diffuse acoustic information as described herein.

In another variation, the elements 604 (FIG. 6) supporting the acoustic diffusers 518 and 520 are adjustable so that the distance between the speakers 512 and 514 and the acoustic diffusers 518 and 520 can be increased or decreased from that shown.

In another variation, the elements 604 (FIG. 6) supporting the acoustic diffusers 518 and 520 include an acoustic reflector which reflects acoustic information traversing a path away from the zone 600 to diffusers 518 and 520.

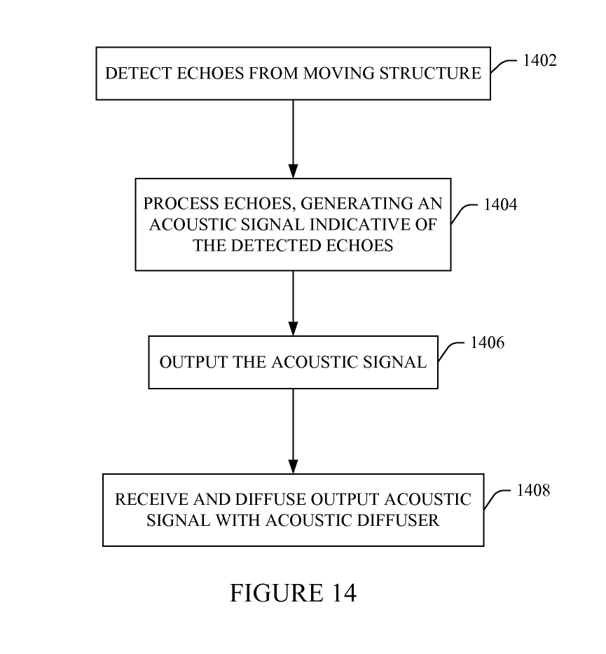

FIG. 14 illustrates a method.

It is to be appreciated that the order of the method acts is provided for explanatory purposes and is not limiting. As such, one or more of the following acts may occur in a different order. Furthermore, one or more of the following acts may be omitted and/or one or more additional acts may be added.

At 1402, ultrasound echoes from moving structure (e.g., blood) are detected by the transducer array 406 of the ultrasound probe 404 of the ultrasound system 400. As discussed herein, the echoes can correspond to ultrasound signals transmitted by the probe 404, under control of the ultrasound console 402, which traverse an imaging field of view and impinge on and reflect off of the moving structure.

At 1404, the echoes are processed, producing acoustic information indicative of a flow of the moving structure.

At 1406, the acoustic information is acoustically presented using an audible output device 430 such as a speaker.

At 1408, the output acoustic information is diffused by an acoustic diffuser 432, which diffuses the acoustic information so that the output acoustic information that traverses a predetermined zone 600 of interest is perceived with a similar or same frequency response and sound pressure level throughout the zone 600.

As discussed above, the zone 600 generally represents the physical area in which the operator of the ultrasound system 400 is located when operating the system 400 to transmit the ultrasound signals and receive the echoes.

It is to be appreciated that the above method may be implemented by one or more processors executing computer executable instructions stored, encoded, embodied, etc. on computer readable storage medium such as computer memory, non-transitory storage, etc. In another instance, the computer executable instructions are additionally or alternatively stored in transitory or signal medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. An ultrasound imaging system, comprising:
receive circuitry configured to receive an ultrasound echo from structure in a medium being scanned with the ultrasound imaging system;
an acoustic processor configured to process the ultrasound echo and generate an acoustic signal indicative of the ultrasound echo;
an acoustic output device configured to acoustically present the acoustic signal; and
an acoustic diffuser configured to receive and diffuse the presented acoustic signal.

2. The system of claim 1, wherein the acoustic diffuser includes at least one element that diffuses acoustic signal such that a frequency response and a sound pressure level of the acoustic signal are substantially the same or the same in a predetermined acoustic zone of interest.

3. The system of claim 2, wherein the zone corresponds to a physical area in which an entity operating the system is located.

4. The system of claim 1, wherein the acoustic output device includes a speaker.

5. The system of claim 1, wherein the acoustic diffuser includes one of a maximum length sequence diffuser, a quadratic-residue diffuser, a primitive root diffuser, an optimized diffuser, or a two dimensional diffuser.

6. The system of claim 1, wherein the acoustic processor includes a Doppler processor configured to process the ultrasound echo based on the Doppler effect and the acoustic signal is a Doppler signal.

7. The system of claim 6, wherein the structure includes a blood cell, the echo corresponds to an ultrasound signal emitted by the system and reflected by the blood cell, and the medium includes a vessel.

8. The system of claim 7, wherein the Doppler signal includes information corresponding to at least one of a flow of the blood cell, a direction of the flow, a change in the direction of flow, or a velocity of the flow.

9. The system of claim 1, wherein the acoustic signal has a frequency in the audible range of the electromagnetic spectrum.

10. The system of claim 1, further comprising:
a visual processor configured to process the ultrasound echo and generate a visual signal indicative of the ultrasound echo; and
a visual output device configured to visually present the visual signal, wherein the visual signal and the acoustic signal are concurrently presented.

11. The system of claim 10, wherein the acoustic output device faces a direction other than a direction in which the visual signal is presented.

12. The system of claim 11, wherein the acoustic diffuser diffuses the acoustic signal at least in the direction in which the visual signal is presented.

13. A method, comprising:
processing, via an acoustic processor, an ultrasound echo and generating an acoustic signal indicative of the ultrasound echo;
outputting the acoustic signal; and
receiving and controllably diffusing, via an acoustic diffuser, the output acoustic signal.

14. The method of claim 13, further comprising:
receiving the ultrasound echo, via receive circuitry, prior to processing and conveying the signal to the acoustic processor for processing.

15. The method of claim 13, further comprising:
diffusing the output acoustic signal such that one or more predetermined acoustic properties of interest of the acoustic signal are substantially the same or the same in a predetermined acoustic zone of interest.

16. The method of claim 15, wherein the zone corresponds to a physical area in which an entity operating the system is located.

17. The method of claim 15, wherein the one or more predetermined acoustic properties of interest includes at least one of a frequency response or a sound pressure level.

18. The method of claim 13, wherein the acoustic diffuser includes one of a maximum length sequence diffuser, a quadratic-residue diffuser, a primitive root diffuser, an optimized diffuser, or a two dimensional diffuser.

19. The method of claim 13, wherein the acoustic signal includes an ultrasound Doppler signal.

20. The method of claim 19, wherein the Doppler signal represents a blood cell flow through a vessel.

21. The method of claim 20, wherein the Doppler signal includes information corresponding to at least one of a flow of the blood cell, a direction of the flow, a change in the direction of flow, or a velocity of the flow.

22. The method of claim 13, wherein the acoustic signal includes a signal with a frequency on an order of about ten kilo-Hertz.

23. The method of claim 13, further comprising:
processing the ultrasound echo and generating an image indicative of the ultrasound echo; and
visually presenting the image.

24. The method of claim 23, wherein the image is visually presented concurrently with the acoustic signal.

25. A method, comprising:
diffusing ultrasound Doppler acoustic information output by an ultrasound imaging system such that at least one of a frequency response and a sound pressure level is the same or substantially the same throughout a predetermined audible information receiving zone.
